(19) United States
(12) Patent Application Publication
(10) Pub. No.: US 2021/0401608 A1
(43) Pub. Date: Dec. 30, 2021

(54) KNEE BRACE

(71) Applicant: Ottobock SE & Co., KGaA, Duderstadt (DE)

(72) Inventors: Heiko Drewitz, Gleichen (DE); Olaf Kroll-Orywahl, Northeim (DE); Klaus Lidolt, Duderstadt (DE); Martin Pusch, Duderstadt (DE); Gordon Siewert, Göttingen (DE); André Mueller, Duderstadt (DE); Markus Tuettemann, Duderstadt (DE); Alexander Schmitt, Kassel (DE); Tim Kruchem, Göttingen (DE); Etienne Overdevest, Göttingen (DE)

(73) Assignee: Ottobock SE & Co., KGaA, Duderstadt (DE)

(21) Appl. No.: 17/289,659

(22) PCT Filed: Oct. 23, 2019
(86) PCT No.: PCT/EP2019/078942

§ 371 Date: Apr. 28, 2021

(30) Foreign Application Priority Data
Oct. 29, 2018 (DE) 10 2018 126 989.7

(51) Int. Cl. A61F 5/01

(52) U.S. Cl. CPC A61F 5/0123; A61F 2005/0174; A61F 2005/0137

(57) ABSTRACT

The invention relates to a knee orthosis with a proximal frame and a distal frame which are connected via a lateral joint and/or a medial join joint such that they can be swiveled, wherein the knee orthosis comprises a proximal pressure element that is arranged exclusively laterally or exclusively medially on the proximal frame by means of a proximal support arm, and a distal pressure element that is arranged exclusively laterally or exclusively medially on the distal frame by means of a distal support arm.

The invention relates to a knee orthosis with a proximal frame and a distal frame, which are connected to each other via a lateral joint and/or a medial joint such they can be swivelled.

Knee orthoses in a wide range of embodiments are known from the prior art and used, for example, to treat osteoarthritis in the knee. They are known either as unilateral knee orthoses or bilateral knee orthoses.

WO 2004/032793 A1 describes a unilateral knee orthosis. It has a proximal section, arranged on the upper leg of the wearer, and a distal section, arranged on the lower leg of the wearer. The two are connected to each other by a joint, which enables movement of the knee. In many cases, such orthoses are used to apply a force to the knee in order to relieve the pain of osteoarthritis. Here, the three-point principle is usually applied. With this principle, the orthosis rests on the wearer's leg at three points where the force is applied. The middle point of the three is located on the knee of the wearer and is often realized by a knee pressure-pad arranged on the side of the respective joint facing the knee. The two other points are located on the upper leg and the lower leg. It is important that the middle force application point lies on a knee side, i.e. medially or laterally, while the two other application points lie on the opposite side.

A unilateral knee orthosis is structurally relatively easy to produce, as there is a connection between the proximal section and the distal section of the orthosis. This is realized by the joint. However, it is disadvantageous that such a unilateral orthosis has only a relatively low degree of stability.

Bilateral knee orthoses are also known from the prior art, for example from DE 60035431 T2. They have a proximal frame and a distal frame which are connected via two joints, namely a lateral joint and a medial joint, such that they can be swivelled. When the knee orthosis is in the mounted state, the lateral joint is located laterally to the knee, while the medial joint is arranged medially to the knee. This configuration enables a higher degree of stability. However, since the force to be applied by way of the upper and lower force application points should be designed to be adjustable, adjustment devices must be provided in the proximal and/or distal frame which enable an adjustment of the force to be applied in the lateral or medial direction. At the same time, these adjustment mechanisms should preferably not place additional or at least excessive loads on the medial joint and/or the lateral joint and yet should still allow movement of the knee. Such orthoses are therefore structurally complex and also expensive.

The invention aims to further develop a knee orthosis in such a way that it is structurally simple to produce and yet allows for the forces to be applied to be adjustable.

The invention solves the task by way of a knee orthosis according to the preamble of claim 1, characterized in that the knee orthosis comprises a proximal pressure element, arranged exclusively laterally or exclusively medially on the proximal frame by means of a proximal support arm, and a distal pressure element, arranged exclusively laterally or exclusively medially on the distal frame by means of a distal support arm.

This means that neither the proximal frame nor the distal frame must allow for swivelling in the frontal plane. This is required with bilateral knee orthoses from the prior art in order to be able to adjust the forces to be applied by the proximal and distal pressure elements. Since the proximal frame and the distal frame of a knee orthosis according to the invention do not have to exhibit this adjustablity, they can be designed to be structurally simple. In particular, no additional forces acting within the frontal plane are exerted on the medial joint and the lateral joint, so that they can also be designed as simple joints that can be constructed with few components, as known from the prior art.

The forces in the lateral or medial direction required for the three-point effect of the bilateral knee orthosis according to the invention are generated by the proximal pressure element, which is arranged on the upper leg of the wearer when the knee orthosis is in the mounted state, and the distal pressure element, which is arranged on the lower leg of the wearer when the knee orthosis is in the mounted state. Each pressure element is fixed to one of the two frames via a support arm. However, this fixing is not bilateral; rather, it is exclusively lateral or exclusively medial. Fixing the support arm of the lateral side of the knee orthosis has the advantage that the connection can be accessed easily and there is no need for additional installation space between the legs of the wearer.

Preferably, the proximal support arm and/or the distal support arm are arranged laterally on the respective frame if the knee orthosis does not have a medial joint but only a lateral joint. If the knee orthosis does not have a lateral joint but only a medial joint, the proximal support arm and/or the distal support arm are preferably arranged medially on the respective frame.

Of course, the reverse is also possible. Where applicable, one of the support arms is arranged laterally and the other support arm medially. This may be the case with a medial joint as well as a lateral joint.

The knee orthosis preferably features a lateral joint as well as a medial joint.

The proximal frame and/or distal frame preferably extend from the medial side to the lateral side of the knee orthosis. This preferably means that, when the knee orthosis is in the mounted state, a part of the respective frame is arranged on the lateral side of the leg and another part on the medial side of the leg.

Preferably, the proximal support arm and the distal support arm are arranged on the same side. They are therefore both arranged laterally or both arranged medially on the respective frame.

It is advantageous if an angle at which the proximal support arm and/or the distal support arm is arranged on the respective frame can be adjusted using an adjustment device. An adjustment of the angle causes the respective pressure element to be displaced laterally or medially, while the length of the support arm stays the same. Said pressure element can thus be adapted to the individual leg shape of the wearer of the knee orthosis. In addition, this renders it possible to adjust the force applied by the pressure element.

As an alternative or in addition to the adjustability of the angle via the adjustment device, the length of the support arm is preferably adjustable. This also allows the pressure element to be displaced laterally or medially, thereby adjusting the force exerted by the pressure element on the wearer's leg.

The angle for the proximal support arm and for the distal support can preferably be adjusted independently of each other. As a result, it is especially easy to follow the individual shape of the wearer's leg. Thus, it is entirely possible that a proximal pressure element must be displaced further laterally than the distal support element in order to exert the same force on the lower leg and the upper leg.

The respective adjustment device can preferably be locked. Preferably, it is infinitely lockable. This allows the angle of the support arm relative to the respective frame, i.e. the proximal support arm relative to the proximal frame and the distal support arm relative to the distal frame, to be infinitely adjusted and the adjustment device can be locked in any of these positions. This can be achieved, for example, by way of a clamping device, a screw device or another adjustment device known in principle from the prior art.

In a structurally particularly simple configuration, the lateral joint and the medial joint allow a swivel movement of the proximal frame relative to the distal frame exclusively in a plane which essentially corresponds to a sagittal plane when the knee orthosis is in the mounted state. In other words, the medial joint and the lateral joint only allow a flexion and extension of the knee, but no movement perpendicular to this plane.

It is advantageous if the proximal support arm and/or the distal support arm are arranged on the respective frame such that they can be detached. As a result, it is easy to replace, for example in order to accommodate changing physical requirements or to repair, replace or clean the support arm.

The proximal pressure element and the distal pressure element are preferably arranged in such a way that they both lie medially or both lie laterally on the wearer's leg when the knee orthosis is in the mounted state. There is preferably a third pressure element on the lateral joint or the medial joint, said third pressure element being arranged on the opposite side of the knee to the proximal pressure element and the distal pressure element. In this way, the three-point effect known in principle can be achieved. As known from the prior art, the middle pressure element may be a knee pressure pad arranged on the medial joint, i.e. the joint located medially of the knee, or on the lateral joint, i.e. the joint located laterally of the knee.

In a preferred configuration, the pressure elements are arranged on the respective support arm such that they can be swivelled. This is especially advantageous if the support arm is arranged on the respective frame such that the angle can be adjusted. This prevents a contact angle of the respective support element on the leg from changing as well.

Preferably, the pressure elements are arranged on the support arms such that they can be detached. This allows the pressure element to be removed, for example in order to clean or repair it, or to replace it with another, more individually suitable support element.

The knee prosthesis preferably features a coupling element which is arranged with a first distal end on the proximal support arm and with a second end on the distal frame, or with the first end on the distal support arm and with the second end on the proximal frame. The fixing points of the two arms of the coupling element are either both situated on the medial side or both situated on the lateral side of the orthosis. It is advantageous if the ends of the coupling element are arranged medially on the respective support arm and frame if the support arm is arranged laterally on the frame, and vice-versa. Therefore, if the support arm is arranged medially on the respective frame, the fixing points for the two ends of the coupling element are located on the lateral side. However, it is of course also possible for the fixing points of the ends of the coupling element and the fixing of the respective support arm to its frame element to be on the same side.

The coupling element is preferably an element that can transmit compressive forces. For example, it may be a corresponding spring or a rigid element, such as a rod.

The coupling element is preferably arranged in such a way that a force applied by the proximal pressure element to the upper leg or by the distal pressure element to the lower leg when the knee orthosis is in the mounted state is varied by the coupling element when the knee is bent. This depends on the force of the pressure element on the support arm that is connected to the coupling element.

For example, if the coupling element is arranged with the first end on the proximal support arm, the second end is of necessity positioned on the distal frame. If the knee is now bent, the force applied by the proximal pressure element on the upper leg is varied by the coupling element and its pressure force transmitting effect when the knee is bent. However, if the coupling element is coupled with the distal support arm and the proximal frame, the force applied to the lower leg by the distal pressure element changes when the knee is bent.

Preferably, this force reaches its maximum at a swivel angle that is greater than 10°, preferably greater than 15°, particularly preferably greater than 17° and less than 30°, preferably less than 25°, particularly preferably less than 22°. It has proven especially advantageous if this swivel angle at which the applied force reaches its maximum is a knee flexion of 22°.

A length of the coupling element can preferably be changed and fixed. This can be achieved, for example, via screw elements, telescopic rods or similar elements. The length of the coupling element can be adjusted following, for example, the release of a locking element or fixing element. This may be a clamping element, a screw connection or another positive-locking element. In this state, the length of the coupling element, for example in the form of a telescopic rod, can be changed. Once the desired length has been set, the fixing or locking element can be fastened again and the length is fixed.

The geometric shape of the coupling element can also be adjustable. For example, rods can be designed to be bent, straight or curved.

The proximal pressure element is preferably located on the side on which the proximal support arm is arranged on the proximal frame. Alternatively or additionally, the distal pressure element is preferably located on the side on which the distal support arm is arranged on the proximal frame. So, if the respective support arm is positioned medially on the respective frame, the respective pressure element is also positioned medially. The support arm therefore does not extend from medial to lateral, but extends only medially. However, if the respective support arm is arranged laterally on the respective frame, the respective distal element is also positioned laterally. The support arm therefore does not extend from lateral to medial, but extends only laterally. Of course, the remaining possibilities are also conceivable.

In the following, an example of an embodiment of the present invention will be explained by way of the attached figures: They show:

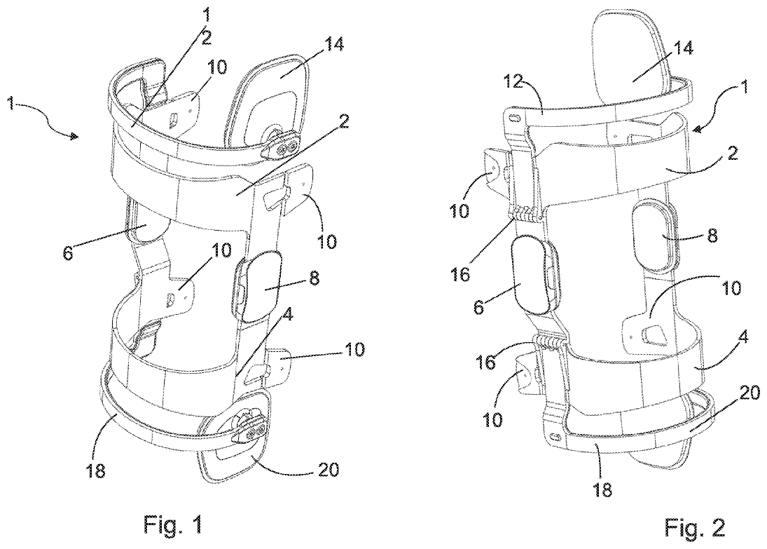

FIGS. 1 and 2—schematic three-dimensional views of an orthosis according to a first example of an embodiment of the present invention,

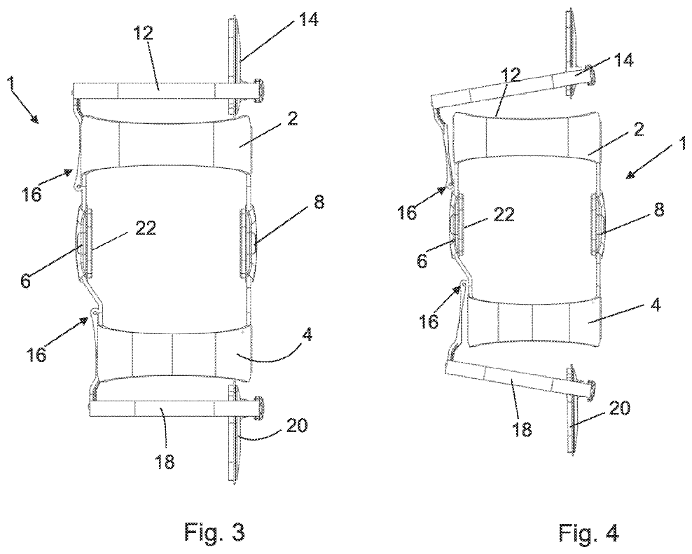

FIGS. 3 and 4—two frontal views of the orthosis depicted in FIGS. 1 and 2,

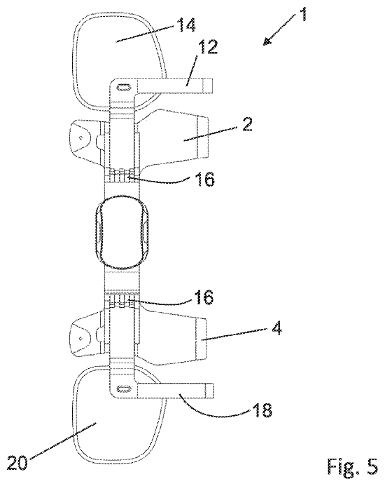

FIG. 5—a side view of the depicted orthosis,

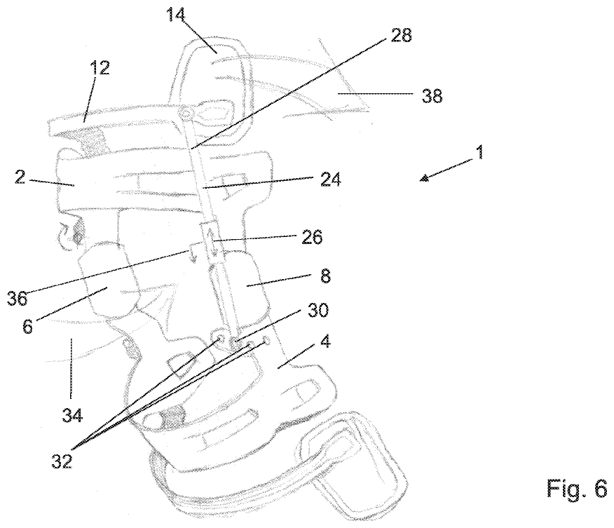

FIG. 6—an orthosis according to a further example of an embodiment of the present invention during flexion,

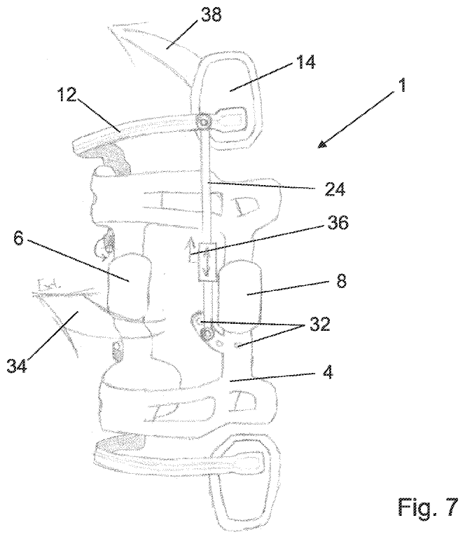

FIG. 7—an orthosis according to a further example of an embodiment of the present invention during extension,

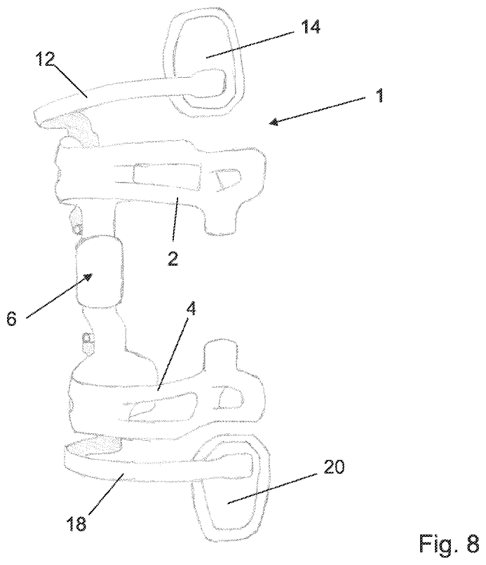

FIG. 8—the schematic view of a unilateral knee orthosis and

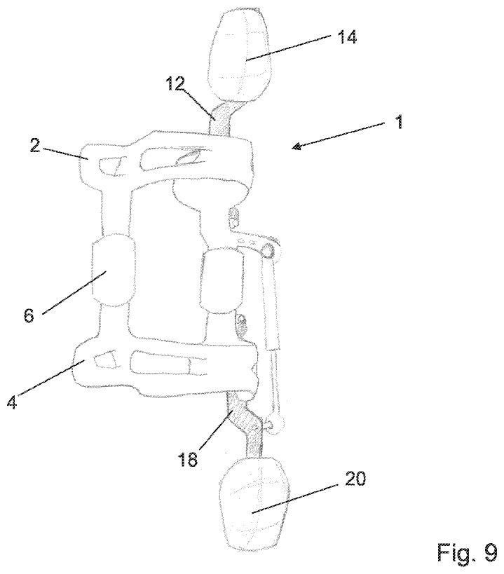

FIG. 9—the schematic view of a further knee orthosis.

FIGS. 1 and 2 depict a knee orthosis 1 according to an example of an embodiment of the present invention. They feature a proximal frame 2 and a distal frame 4. The proximal frame 2 and the distal frame 4 are connected to each other by a lateral joint 6 and a medial joint 8 such that they can be swivelled. The lateral joint 6 and the medial joint 8 each allow a movement along a sagittal plane. The flexion and extension of a knee is thus not impeded.

The proximal frame 2 and the distal frame 4 both feature fastening clips 10, which may be fixed to a belt, in order to fix the knee orthosis 1 to the leg of the wearer.

The knee orthosis 1 also has a proximal support arm 12, on which a proximal pressure element 14 is situated. The proximal support arm 12 is arranged laterally on the proximal frame 2 via an adjustment device 16.

A distal support arm 18 is arranged on the distal frame 4 via a second adjustment device 16, wherein a distal pressure element 20 is located on said support arm.

The proximal pressure element 14 and the distal pressure element 20 are cushions or pressure pads that rest on the leg of the wearer.

FIGS. 3 and 4 show a frontal view of the knee orthosis 1 from FIGS. 1 and 2. The proximal frame 2 is connected to the distal frame 4 via the lateral joint 6 and the medial joint 8. FIGS. 3 and 4 depict two different angular settings of the adjustment device 16. While the proximal support arm 12 and the distal support arm 18 in FIG. 3 extend horizontally, they exhibit a relatively strong incline in FIG. 4. This results in the proximal pressure element 14 and the distal pressure element 20 in FIG. 4 being displaced laterally, i.e. to the left in the view depicted, in comparison to FIG. 3. A considerably stronger force is exerted in the lateral direction on a leg that is in the knee orthosis 1 in FIG. 4 than in FIG. 3. Of course, it is also possible to select a proximal support arm 12 and a distal support arm 18 that are designed in such a way that the proximal pressure element 14 and the distal pressure element 20 are located on the medial side. In the example of an embodiment shown, a third pressure element 22 is located on the side of the lateral joint 6 that faces the knee.

FIG. 5 depicts a lateral side view of the knee orthosis 1. The adjustment devices 16 can be seen, by means of which the proximal support arm 12 with the proximal pressure element 14 located on it and the distal support arm 18 with the distal pressure element 20 located on it are arranged on the respective proximal frame 2 and the distal frame 4.

FIG. 6 depicts the knee orthosis 1 in which, unlike the knee orthoses shown thus far, a coupling element 24 is arranged between the distal frame 4 and the proximal support arm 12. In the example of an embodiment shown, the coupling element 24 is a length-adjustable rod that features a length adjustment device 26. By way of this length adjustment device 26, it is possible to change the length of the coupling device 24 and set the desired dimension, and to fix the length again after adjustment so that the main task of the coupling element, namely to transmit tensile or compressive forces, can be fulfilled.

A first end 28 of the coupling element 24 is arranged on the proximal support arm 12. A second end 30 opposite the first end 28 is positioned on the distal frame 4. This may occur in various positions. In the example of an embodiment shown, the distal frame 4 features four holes 32 for this purpose, in which the second end 30 of the coupling element 24 can be fixed.

FIG. 6 depicts the effect of the coupling element 24 during a flexion, i.e. a bending of the knee. This is shown by the first arrow 34. The swivel movement of the distal frame 4 relative to the proximal frame 2 results in the second end 30 and thus the coupling element 24 in FIG. 6 being moved downwards, which is shown via the second arrow 36. This tensile force, which is transmitted to the proximal support arm 12 via the coupling element 24, causes the proximal pressure element 14 to move away from the wearer's leg, or at least reduces the force exerted by the proximal pressure element 14. This is shown by the third arrow 38.

FIG. 7 depicts the orthosis from FIG. 6 during the extension movement. In this case, the coupling element 24 is also arranged on the proximal support arm 12 and the distal frame 4. The extension is shown by the first arrow 34. Due to this swivel movement, the coupling element 24 is moved upwards in accordance with the second arrow 36, which causes a compressive force to be transmitted to the proximal support arm. This causes the proximal pressure element 14 to move towards the leg, or at least increases the applied force. This is shown by the third arrow 38.

The force path can be influenced by selecting an articulation point of the first end 28 and/or the second end 30 of the coupling element 24. If the second end 30 of the coupling element 24 is inserted into or fixed to the right-hand hole of the holes 32, for example, the coupling element 24 does not extend in front of the swivel axis of the lateral joint 6 and the medial joint 8, as shown in FIGS. 6 and 7; rather, it extends behind the corresponding swivel axis. As a result, it is possible, for example, for a compressive force rather than a tensile force to be transmitted through the coupling element 24 to the proximal support arm 12 when the joint is flexed, as shown in FIG. 6. By carefully selecting an articulation point, such as the position of one of the holes 32, the force path can be adjusted almost arbitrarily. In particular, it can be ensured that the force applied by the proximal pressure element 14, for example during a flexion of the knee joint, initially increases and then decreases again when a maximum swivel angle at which the force reaches its maximum is exceeded.

The same applies for a knee orthosis 1 in which the coupling element 24 is fixed to the proximal frame 2 and the distal support arm 18. In this case, the force applied by the distal pressure element 20 when the knee is flexed or extended is varied.

FIG. 8 depicts a knee orthosis 1 that only features a lateral joint 6 and no medial joint 8. The proximal frame 2 and the distal frame 4 still extend from the medial side to the lateral side of the knee orthosis 1 and in the mounted state, not depicted in FIG. 8, also from the medial side to the lateral side of the leg and therefore the knee. The proximal support arm 12, on which the proximal pressure element 14 is located, is arranged on the lateral side of the proximal frame 2. In the same way, the distal support arm 18, on which the distal support element 20 is located, is also arranged on the lateral side of the distal frame 4.

FIG. 9 depicts a further embodiment of a knee orthosis 1 in which the proximal support arm 12 and the distal support arm 18 are arranged on the same side, for example laterally, on the proximal frame 2 or the distal frame 4. The proximal pressure element 14 and the distal pressure element 20 are found on the same side, i.e. also laterally in the example shown.

Reference list

1 knee orthosis

2 proximal frame

4 distal frame

6 lateral joint

8 medial joint

10 fastening clip

12 proximal support arm

14 proximal pressure element

16 adjustment device

18 distal support arm

20 distal pressure element

22 third pressure element

24 coupling element

26 length adjustment device

28 first end

30 second end

32 hole

34 first arrow

36 second arrow

38 third arrow

1. A knee orthosis with
a. a proximal frame and
b. a distal frame,
which are connected via a lateral joint and/or a medial joint such that they can be swiveled,
wherein
the knee orthosis comprises a proximal pressure element, which is arranged exclusively laterally or exclusively medially on the proximal frame by means of a proximal support arm, and
a distal pressure element, which is arranged exclusively laterally or exclusively medially on the distal frame by means of a distal support arm.

2. The knee orthosis according to claim 1, wherein the proximal support arm and the distal support arm are arranged on the same side on the respective frame.

3. The knee orthosis according to claim 1, wherein an angle at which the proximal support arm and/or the distal support arm is arranged on the respective frame can each be adjusted using an adjustment device.

4. The knee orthosis according to claim 3, wherein the angle of the proximal support arm and the angle of the distal support arm can be adjusted independently of each other.

5. The knee orthosis according to claim 3, wherein the adjustment devices can be locked.

6. The knee orthosis according to claim 1, wherein the lateral joint and the medial joint allow a swivel movement of the proximal frame relative to the distal frame exclusively in a plane which essentially corresponds to a sagittal plane when the knee orthosis is in the mounted state.

7. The knee orthosis according to claim 1, wherein the proximal support arm and/or the distal support arm are arranged on the respective frame such that they can be detached.

8. The knee orthosis according to claim 1, wherein the proximal pressure element and the distal pressure element are arranged in such a way that they both lie medially or they both lie laterally on a leg of the wearer when the knee orthosis is in the mounted state.

9. The knee orthosis according to claim 1, wherein the pressure elements are arranged on the support arms such that they can be swiveled.

10. The knee orthosis according to claim 1, wherein the pressure elements are arranged on the support arms such that they can be detached.

11. The knee orthosis according to claim 1, wherein the knee orthosis features a coupling element which is arranged with a first end on the proximal support arm and with a second end on the distal frame , or with the first end on the distal support arm and with the second end on the proximal frame.

12. The knee orthosis according to claim 11, wherein the coupling element is arranged in such a way that a force applied by the proximal pressure element to the upper leg or by the distal pressure element to the lower leg when the knee orthosis is in the mounted state is varied by the coupling element when the knee is bent.

13. The knee orthosis according to claim 12, wherein the force reaches its maximum at a swivel angle that is greater than 10°, preferably greater than 15°, particularly preferably greater than 17° and less than 30°, preferably less than 25°, particularly preferably less than 22°.

14. The knee orthosis according to claim 13, wherein the swivel angle is 20°.

15. The knee orthosis according to claim 11, wherein a length of the coupling element can be changed and fixed.

16. The knee orthosis according to claim 1, wherein the proximal pressure element is located on the side on which the proximal support arm is arranged on the proximal frame and/or that the distal element is located on the side on which the distal support arm is arranged on the proximal frame.

17. A knee orthosis comprising:
a proximal frame and a distal frame which are connected via a lateral joint and/or a medial joint such that they can be swiveled;
a proximal pressure element which is arranged exclusively laterally or exclusively medially on the proximal frame at an angle by means of a proximal support arm; and
a distal pressure element which is arranged exclusively laterally or exclusively medially on the distal frame at an angle by means of a distal support arm;
wherein the proximal support arm and the distal support arm are arranged on the same sides of the proximal or distal frame to which they are mounted, and wherein the angle at which they are arranged can be independently adjusted using an adjustment device.

18. The knee orthosis of claim 17, wherein the adjustment devices can be locked.

19. The knee orthosis according to claim 18 wherein the lateral joint and the medial joint allow a swivel movement of the proximal frame relative to the distal frame exclusively in a plane which essentially corresponds to a sagittal plane when the knee orthosis is in a mounted state.

20. A knee orthosis comprising:
a proximal frame and a distal frame which are connected via a lateral joint and/or a medial joint such that they can be swiveled;
a proximal pressure element which is arranged exclusively laterally or exclusively medially on the proximal frame at an angle by means of a detachably-mounted proximal support arm; and
a distal pressure element which is arranged exclusively laterally or exclusively medially on the distal frame at an angle by means of a detachably-mounted distal support arm;
wherein the proximal support arm and the distal support arm are arranged on the same sides of the proximal or distal frame to which they are mounted, and wherein the angle at which they are arranged can be independently adjusted using a lockable adjustment device.
